(19) United States
(12) Patent Application Publication
(10) Pub. No.: US 2025/0275839 A1
(43) Pub. Date: Sep. 4, 2025

(54) BLANK AND METHOD FOR MANUFACTURING THE SAME

(71) Applicant: DEGUDENT GMBH, Hanau (DE)

(72) Inventors: Lothar VOELKL, Hanau (DE); Stefan FECHER, Hanau (DE); Martin KUTZNER, Hanau (DE); Sven GIESE, Hanau (DE); Christian KOHLER, Hanau (DE)

(21) Appl. No.: 18/858,992

(22) PCT Filed: Apr. 4, 2023
(86) PCT No.: PCT/EP2023/058748

§ 371 Date: Oct. 22, 2024

(30) Foreign Application Priority Data
Apr. 27, 2022 (EP) 22170325.9

(51) Int. Cl. A61C 13/00; A61C 13/08; A61K 6/818; C04B 35/48; C04B 35/64

(52) U.S. Cl. CPC A61C 13/0022; A61C 13/082; A61K 6/818; C04B 35/48; C04B 35/64; C04B 2235/3224; C04B 2235/3246; C04B 2235/604; C04B 2235/765

(57) ABSTRACT

The invention relates to a blank and a method for producing a blank for use in the production of a dental product consisting of or containing ZrO₂ stabilised with Gd₂O₃ as the main stabiliser with a content of between 2 and 6 mol %, relative to the content of ZrO₂ and at least one oxide from the group Y, Yb, Dy, Nd, Ca, Ce, Mg, Sm, Er, Tb, La as a secondary stabiliser with a content of between 0.05 and 2.0 mol %, relative to the content of ZrO₂.

The invention relates to a blank for use in producing a dental product, such as a dental restoration, veneer or implant component, consisting of or containing zirconium dioxide (ZrO₂) stabilised with at least gadolinium(III) oxide (Gd₂O₃).

A corresponding blank can be found in EP 2 956 427 B1. The content of tetragonal zirconium dioxide is between 94 vol % and 96 vol % in order to be able to achieve the object of providing a dental ceramic with good mechanical properties.

WO 99/47065 A1 discloses a method for producing a dental prosthesis that can be fitted on a pre-prepared tooth stump and is based on a zirconium dioxide blank. The blank consists of a pre-sintered zirconium dioxide disc, from which a mould corresponding to the dental prosthesis is machined, taking into account the shrinkage behaviour during full or final sintering. The starting powder may contain colouring elements in the form of oxides.

An inorganic-inorganic composite material and a method for its production are known from WO 2005/070322 A1. To produce the composite material, an open-pored, crystalline oxide ceramic moulding is made from an oxide ceramic powder of ZrO₂ (zirconium dioxide) after shaping processing and pre-sintering, an infiltration material is applied to this under vacuum at room temperature and the oxide ceramic is sintered at air atmosphere and ambient pressure to compact the inorganic-inorganic composite material. These measures are intended to result in an improved aesthetic effect.

WO 2015/199018 A1 discloses a coloured translucent zirconium dioxide body which consists of yttrium oxide-stabilised zirconium dioxide, erbium oxide, iron oxide, cobalt oxide and aluminium oxide.

A blank made of zirconium dioxide is known from U.S. Pat. No. 8,936,845 B2, which is used for the production of dentures and consists of a plurality of layers of different chemical compositions. The individual layers have different proportions of yttrium oxide.

A zirconium dioxide blank for the production of dental products according to WO 2014/062375 A1 has at least two material regions that have different proportions of the tetragonal and cubic crystal phases, wherein the quotient is greater in one of the regions and the quotient is less than 1 in the other region.

EP 2 371 344 A1 relates to a ceramic body that, starting from the surface and down to a desired depth, is enriched with a stabilising agent.

The object of the present invention is to achieve, among other things, that the blank has the strength required for use as a dental product, in particular as a restoration or implant, while at the same time showing desired optical properties in order to meet aesthetic requirements. The blanks intended for dental restorations should have an appearance that corresponds to that of natural teeth.

To achieve one or more aspects, the invention provides that the zirconium dioxide is stabilised with Gd₂O₃ as the main stabiliser with a content of between 2 and 6 mol % relative to the content of ZrO₂ and with at least one oxide from the group Y, Yb, Dy, Nd, Ca, Ce, Mg, Sm, Er, Tb, La as a secondary stabiliser with a content of between 0.05 and 2.0 mol % relative to the content of ZrO₂.

Surprisingly, it has been found that when gadolinium(III) oxide is used as the main stabiliser, whereby high fracture toughness can be achieved with good optical properties at the same time, at least one other stabiliser is used as a secondary stabiliser, the fracture toughness is not noticeably reduced, but at the same time the strength is increased. Increasing the stabiliser content also improves the optical properties.

In particular, it is provided that the blank contains at least one colouring oxide from the group Pr, Er, Fe. Co, Ni, Ti, V, Cr, Cu, Mn, Tb.

The invention preferably provides that the proportion of Gd₂O₃ is between 2 and 5 mol %, particularly preferably between 2.5 and 4 mol %, very particularly preferably between 3 and 4 mol %, relative to the content of ZrO₂.

It should also be emphasised that the tetragonal crystal phase of the zirconium dioxide of the blank is between 40 vol % and 80 vol %, in particular between 45 vol % and 75 vol %. In contrast to blanks that contain Gd₂O₃ as the main stabiliser, the proportion of the tetragonal phase of the stabilised zirconium dioxide is deliberately reduced in order to obtain a relatively high proportion of the cubic crystal phase in order to achieve the desired translucency. Irrespective of this, however, the required strength or fracture toughness and flexural strength are given in order to meet the mechanical requirements that are placed on a restoration or implant component.

Due to the mandatory content of at least one colouring oxide, an optical appearance can be achieved that satisfies aesthetic requirements. The colouring of implant components presents the advantage that if the implant becomes visible due to changes in the gums, this is hardly noticeable in comparison to implants made of titanium, for example.

In particular, the secondary stabiliser content should be between 0.05 and 2.0 mol %, in particular between 0.2 and 0.8 mol %, relative to the zirconium dioxide content if a single secondary stabiliser or if several secondary stabilisers are present.

With regard to the colour effect to be achieved, it is provided in particular that the proportion of the colouring oxide or the colouring oxides in wt % of the blank is max. 1.5 wt %.

If it is possible to provide a single-layer and thus single-colour blank according to the teaching according to the invention. it is provided in particular that the blank has at least two regions with compositions that differ from one another.

The total content of the stabiliser(s) can be the same in each region.

In other words, the sum of the contents of the stabilisers in each layer can be the same, wherein the content of the colouring oxide(s) in the layers differ from one another.

However, there is also the possibility that the total content and/or the stabilisers are different in the regions. If Y₂O₃ is used as the stabiliser, the content of this in the layers can be the same.

As mentioned, the additional stabiliser(s) are referred to as secondary stabilisers, as the stabiliser Gd₂O₃ is the main stabiliser, i.e. it is primarily used for stabilising the tetragonal crystal phase in the zirconium dioxide.

The ratio of the main stabiliser Gd₂O₃ to the secondary stabiliser or the sum of the stabilising secondary stabilisers is thus 1:1 to 1:120, preferably 1:2 to 1:40, particularly preferably 1:4 to 1:10, particularly preferably 1:5 to 1:7.

In order to achieve desired fluorescence properties, in particular for a dental restoration to be produced from the blank, it is provided that the blank or a region of the blank contains at least one element that produces a fluorescence effect, in particular at least one oxide from the group Bi, Tb, Tm, Pr, with a proportion in wt % of between 0.005 and 2.0 wt %, preferably between 0.005 and 0.5 wt %.

In order to be able to produce a dental restoration from a blank, such as a framework, bridge, crown, partial crown, which can be made available without costly reworking, but at the same time satisfies the desired aesthetic requirements and has the required strength in heavily stressed regions, it is in particular proposed that a first region has a cavity within which the second region, which has a composition that differs from the first region, extends.

The first region can have a plurality of cavities, optionally with different internal geometries, in which a plurality of second regions extend and in that the first region has a greater translucency than the second region, the strength of which is greater than that of the first region.

In particular, it is provided that the blank is multi-layered, comprising at least one bottom layer and one top layer of different compositions, wherein the layers contain at least one first colouring oxide, the proportion of which in the bottom layer having the first colouring oxide is lower than in the top layer having the first colouring oxide.

Irrespective of this, the blank should have at least three layers, wherein the middle layer running between an upper and a lower layer consists of a material of the upper layer and the lower layer.

In order to meet aesthetic requirements in particular, a further proposal of the invention provides that the first colouring oxide is at least one oxide from the group Co, Mn, Ni, Cr, in particular Co₂O₃ or MnO₂ or a mixture thereof.

The invention also relates to a method for producing a blank intended for producing a dental product, in particular a dental restoration, veneer, implant component, in which powdered ceramic material containing zirconium dioxide stabilised with at least one stabiliser is pressed and then subjected to at least one heat treatment, which is thereby characterised in that the zirconium dioxide is stabilised with gadolinium(III) oxide (Gd₂O₃) as the main stabiliser with a content in mol % relative to the content of zirconium dioxide of between 2 and 6 mol % and at least one oxide from the group Y, Yb, Dy, Nd, Ca, Ce, Mg, Sm, Er, Tb, La as a secondary stabiliser with a content of between 0.05 and 2.0 mol % relative to the content of ZrO₂.

At least one colouring oxide from the group Pr, Er, Fe, Co, Ni, Ti, V, Cr, Cu, Mn, Tb is preferably added to the ceramic material.

In particular, the invention is characterised by a method for producing a blank from a ceramic material, wherein at least two layers of ceramic material consisting of the mixtures described above are introduced into a die in layers, wherein the layers can have different compositions, and then after introduction of the layers, these are pressed and then sintered, wherein after the introduction of the first layer, this is structured on the surface in such a way that the first layer, viewed along its surface, differs in height in some regions, i.e. does not have a uniform filling height, and then a second layer, which differs in composition from the first layer, is introduced into the mould.

Alternatively, there is the possibility that, after the first layer has been applied, an intermediate layer made of a ceramic material from a mixture according to the invention is filled into the die that differs from that of the first layer, that the material of the first layer is mixed with the material of the intermediate layer, and that the second layer is then introduced into the die. It is provided in particular that the material of the intermediate layer is mixed with that of the first layer, starting from the free surface of the intermediate layer, over a height that corresponds to twice or approximately twice the height of the intermediate layer. Furthermore, it is provided in particular that the material used for the intermediate layer is one that matches that of the second layer.

According to the invention, according to the first alternative, a first layer of pourable powdered material is introduced into a die. After the material has been introduced, the surface is smoothed in order to then form a structure in such a way that elevations and valleys result, which in particular run parallel to one another, but in particular run concentrically or parallel to one another. For this purpose, it is provided in particular that the structure is formed by an element moving, in particular rotating, relative to the first layer, which structures the first layer in its surface region, in particular with a section formed like a wave, comb or sawtooth. The surface is “raked”, so to speak, in order to form the structure, i.e. the alternating elevations and valleys.

In particular, it is provided that the structure is introduced in such a way that the volume of the elevations is the same or approximately the same as the volume of the depressions or valleys.

The sawtooth-like element should preferably have V-shaped teeth that are symmetrical and the flanks of which enclose an angle of between 15° and 45°. The distance between consecutive teeth, i.e. the distance from tip to tip, should be between 1 and 4 mm, preferably between 1 mm and 3 mm.

The pourable, powdery second ceramic material is then introduced into the mould, which increases in quantity starting from the depressions in the structure formed by the valleys, so that as a result there is a quasi-continuous increase in the proportion of the second layer over the height of the elevations. After the surface has been smoothed, the layers are pressed.

This is followed by pre-sintering at a temperature between 700° C. and 1100° C., in particular in a range between 800° C. and 1000° C. over a period of e.g. 100 minutes to 150 minutes. The blank thus produced is then machined e.g. by milling and/or grinding to produce a desired dental restoration, which is then sintered.

The sintering takes place e.g. for a time between 10 minutes and 250 minutes in a temperature range between 1200° C. and 1600° C.

In particular, sintering should be carried out in the temperature range between 1400° C. and 1500°° C., preferably between 1400° C. and 1450° C.

The temperatures and times for pre-sintering or sintering explained above apply to different layer shapes, layer sequences and different numbers of layers, although of course this also includes the production of a blank that consists of a uniform material, i.e. does not consist of layers or regions of ceramic material, which have different compositions with regard to the starting raw materials.

Penetrating the layers has the advantage that different physical and optical properties can be achieved over the height of the blank. Thus, if the first layer is coloured to the required extent, a tooth-coloured marginal region can be achieved after sintering, i.e. finishing or fully sintering, which is continuously decreasing in intensity over the transition region created by the interpenetrating first and second layer materials. The dental restoration is then produced from the blank, in particular by milling, taking into account the course of the layer, wherein the dental restoration is “nested” into the blank in such a way that the cutting edge of the tooth runs in the region of the second layer.

Due to the teaching according to the invention, there is a continuous transition between the layers, such that the colour or translucency decreases or increases continuously.

The use of Gd₂O₃ as the main stabiliser results in particular in the advantage that high edge stability of the dental product can be achieved. Compared to ZrO₂ stabilised with Y₂O₃ as the main stabiliser, the wall thickness can be up to 20% thinner.

It is preferably provided that the possibility of mixing the layer materials is opened up by rotating a structuring element, in particular about an axis running along the longitudinal axis of the mould, in order to create the structure, which can also be described as wave-like or sawtooth-like, by displacing material on the surface to achieve the first layer. There is also the possibility of forming the structure by means of a pressure element acting in the direction of the surface on the first layer, which in particular has elevations running in its surface with depressions running therebetween, so that the negative shape is imprinted into the surface of the first layer by the element that can also be called a plunger. Then—as explained above—the ceramic material of the second layer is filled in, then smoothed to press only the layers together and then pre-sinter the pressed part.

The subject matter of the invention is also a dental product in the form of a dental restoration, in particular a crown, partial crown or bridge, wherein the restoration, viewed in the direction of the tooth axis, consists of at least a first layer running on the root side and a second layer running on the incisal side, wherein the strength of the first layer is greater than that of the second layer and the translucency of the second layer is greater than that of the first layer.

The layers should contain a first colouring oxide, in particular at least one oxide from the group Co, Mn, Ni, Cr, preferably Co₃O₄ or MnO₂ or a mixture thereof, wherein the proportion in the first layer is lower than in the second layer.

Furthermore, it is proposed that the dental product is a one-piece implant system consisting of an implant and abutment, although this can also be in two parts in the usual way.

Further details, advantages and features of the invention result not only from the claims, the features to be extracted from them-individually and/or in combination-but also from the following description of preferred exemplary embodiment to be taken from the drawings as well as their explanations.

### IN THE DRAWINGS

FIG. 1 shows a schematic diagram of a device for producing a blank,

FIG. 2 shows a schematic diagram of a device to illustrate the method steps for producing a green body,

FIG. 3 shows a blank with regions of different material properties,

FIG. 4 shows a further embodiment of a blank with regions of different material properties,

FIG. 5 shows a schematic diagram of a blank with a dental prosthesis to be machined therefrom,

FIG. 6 shows a further embodiment of a blank with several regions of different material properties,

FIG. 7 shows a schematic diagram of a device with the aid of which method steps are to be carried out for producing a green body, and

FIG. 8 shows a further schematic diagram for explaining production method steps.

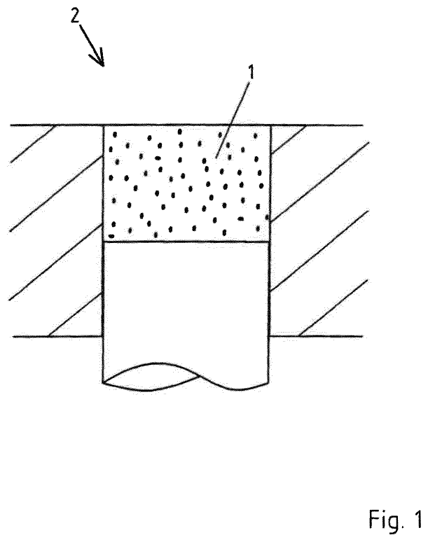

In order to produce a blank, a powdery ceramic material is first produced, the main component of which is stabilised zirconium dioxide, which is stabilised with Gd₂O₃ as the main stabiliser and one or more secondary stabilisers. At least one oxide from the group Y, Yb, Dy, Nd, Ca, Ce, Mg, Sm, Er, Tb, La with a content between 0.05 and 2.0 mol % relative to the content of ZrO₂ is considered as a secondary stabiliser.

The ratio of main stabiliser to secondary stabiliser or the sum of the stabilising secondary stabilisers, in mol % relative to zirconium dioxide, should be 1:1 to 1:120, preferably 1: 2 to 1:40. particularly preferably 1:4 to 1:10, particularly preferably 1:5 to 1:7.

The total content of the stabiliser(s) should be between 2 mol % and 8 mol %.

At least one colouring oxide is also added, the proportion by weight of which in the ceramic material should not be more than 1.5 wt %. In particular, Fe₂O₃, Er₂O₃, Co₃O₄ or Tb₂O₃ or a mixture of two or more of these oxides is selected as the colouring oxide.

The mixture also contains HfO₂<3.0 wt %, Al₂O₃<0.3 wt %, unavoidable admixtures for technical reasons ≤0.2. If necessary, an element producing a fluorescence effect, such as bismuth or thulium, can be added, the respective oxide content of which should be 0.005 to 2.0 wt %, in particular 0.005 to 0.5 wt %.

A correspondingly prepared mixture 1 is poured into a mould or die 2 and pressed. After the green body produced in this way has been removed from the mould, it is then subjected to a first heat treatment in the range between 800° C. and 1000° C. over a period of between 100 minutes and 150 minutes. The first step is debinding if a binder has been added to the starting mixture and then pre-sintering. The pre-sintered blank is then machined, for example to carve out an artificial tooth. Sintering then takes place. i.e. fully sintering in the temperature range between preferably 1400° C. and 1500° C. wherein values between 1400° C. and 1450° C. are particularly noteworthy. The sintering process is carried out over a period of between 10 minutes and 250 minutes. In this way a monochromatic dental product is produced. This can be a dental restoration or, for example, with particular emphasis, an implant system, which consists of an implant and an abutment and can therefore be produced in one piece or (as is usual) can be in two parts.

The use of Gd₂O₃ as the main stabiliser results in the advantage that, contrary to proposals from the prior art, the proportion of the tetragonal crystal phase can be relatively small, specifically between 40 vol % and 80 vol %, preferably between 45 vol % and 75 vol %, such that, as a result, the proportion of the cubic crystal phase is relatively high and a desired translucency can thus be achieved. Irrespective of the relatively small proportion of the tetragonal crystal phase, a corresponding sintered blank has the strength, fracture toughness and hardness required for use in the dental field. Experiments have shown that the biaxial strength measured according to ISO 6872 with Gd₂O₃-stabilised zirconium dioxide, wherein the content of Gd₂O₃ is 3 mol % relative to the content of zirconium dioxide, depending on sintering temperatures, is in the range between 750 MPa to 1000 MPa and in the case of zirconium dioxide stabilised with gadolinium(III) oxide as the main stabiliser (3 mol %), with ytterbium oxide as the secondary stabiliser (0.5 mol %), is in the range between 750 MPa and 850 MPa—values that are only achievable with yttrium oxide-stabilised ZrO₂ if the tetragonal crystal phase was more than 90 vol %.

Zirconium dioxide stabilised with 3 mol % Gd₂O₃ resulted in a fracture toughness K_{IC}, measured by the chevron notch beam method, in the unit MPa m0.5 in the range between 10 and 12 at sintering temperatures between 1350° C. and 1450° C. A mixture of stabilisers consisting of 3 mol % gadolinium(III) oxide as the main stabiliser and 0.5 mol % ytterbium oxide as the secondary stabiliser resulted in K_{IC} values of between 8 and 10.5 MPa m⁰⁵. In contrast, a K_{IC} value of 4-5 MPa m^{0.5} results for Y₂O₃ (3 mol %) stabilised ZrO₂.

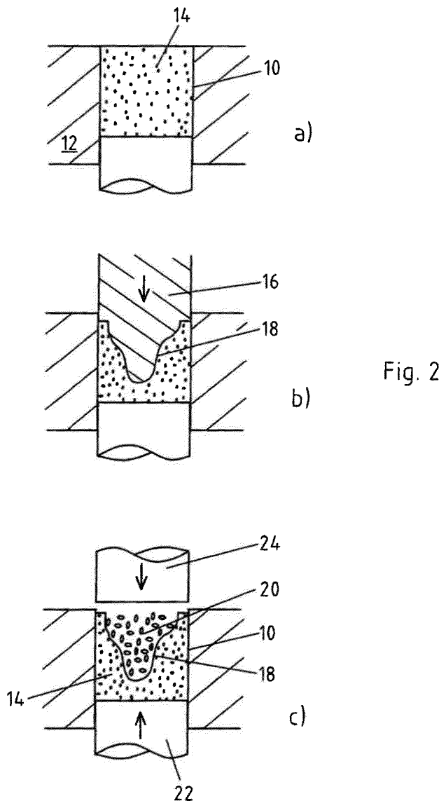

With reference to FIGS. 2 to 7, further aspects shaping the invention are to be clarified in order to produce monolithic products, in particular dental restorations such as crowns, partial crowns or bridges, wherein the blanks have regions close to the contour in relation to the restorations to be produced.

It should be explained that blanks can be produced that have regions of ceramic material with different compositions and thus properties in order to achieve desired optical and mechanical properties. The advantage of the monolithic denture is that it can in principle be used immediately after it has been machined from the blank and then sintered, without, for example, the need for a cutting edge to be applied and fired by hand.

Desired strength values can be set in a targeted manner. The same applies to colour, translucent and fluorescent properties.

According to FIG. 2a, a powder of a first composition is filled into a die 10, which powder is to be used, for example, as a cutting material. The powder can contain binders. The powder has a composition as explained above by way of example.

After filling the material 14 into the die 10, an open cavity 18 is formed by means of a press plunger 16, wherein the material itself is not pressed in principle. Rather, material is displaced or slightly compressed. After the cavity has been formed (FIG. 2b), the press plunger 16 is removed in order to fill the cavity with a second ceramic material 20, which can differ from the first ceramic material 14 in that the content of the stabiliser oxides present in the materials is the same in each material, but the colouring oxides added differ from each other, either in element, proportion by weight, or a combination thereof.

The same applies if components are added that are intended to achieve fluorescence properties.

However, there is also the possibility that each material contains different amounts of stabilisers, but at least each material contains Gd₂O₃ as the main stabiliser and a further secondary stabiliser.

After filling the second ceramic material 20 into the cavity 18 (FIG. 2c), the materials 14, 20 or the layers or regions formed therefrom are then pressed in the die 10 of the press 12 by means of a lower and/or upper plunger 22, 24 to achieve compaction.

After pressing, the green body 28 has a density of approximately 3 g/cm³ (FIG. 3). The pressing can be carried out in the desired customary ranges with a pressure of, for example, between 1000 and 2000 bar.

As can be seen from FIG. 3, a second cavity 26 can be machined into the second material 20, for example by milling, after it has been compressed by means of the press plunger 22, 24 or, optionally, after pre-sintering.

However, it is also possible to form a corresponding second cavity 26 in the material 20 according to FIG. 2c, which completely fills the bottom-side open cavity 18, by means of a press plunger (not shown) in order to fill in a further material that should differ in composition from the material previously filled into the die.

Irrespective of whether the second cavity 26 is present or not, the blank 28 is pre-sintered after pressing, in particular in the range between 800° C. and 1000° C. over a period of between 100 minutes and 150 minutes. If a binder is present in the material, first debinding and then pre-sintering occurs.

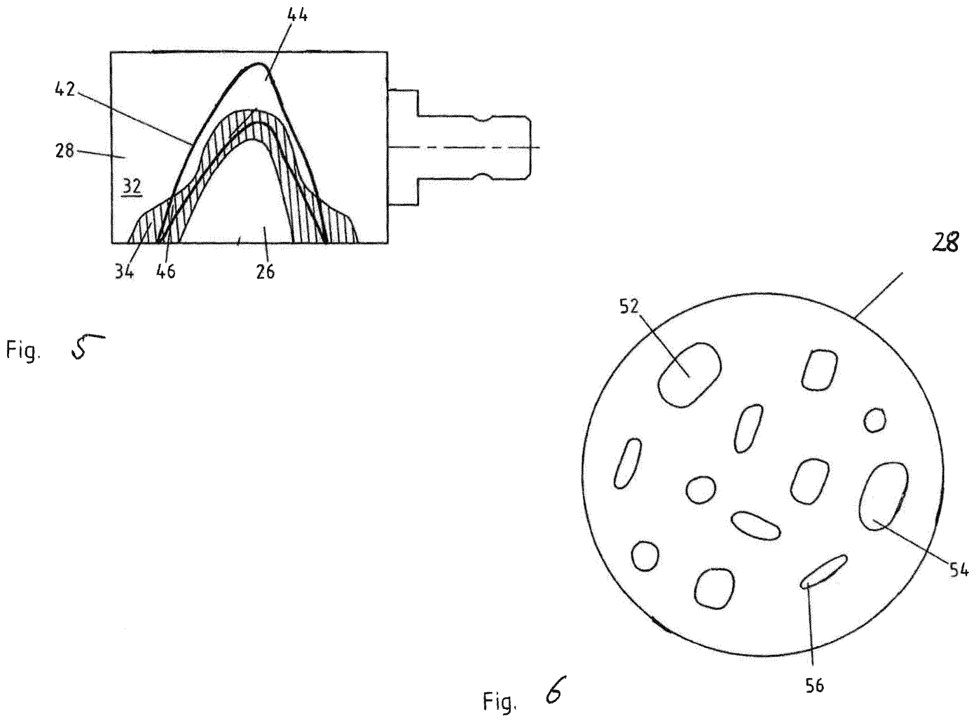

The blank is provided with a holder in order to carry out machining, for example by milling and/or grinding, so that a desired dental product, such as a tooth, can be machined from the blank 28, as will be explained with reference to FIG. 5.

The tooth to be produced is preferably placed at least virtually in the blank 28 in such a way that the cutting region runs in the region 32 formed by the first ceramic material 14 and the dentine region runs in sections in the second region 34 formed by the second ceramic material 20. Then, taking this data into account, the blank 28 is processed, wherein the shrinkage behaviour of the material is taken into account.

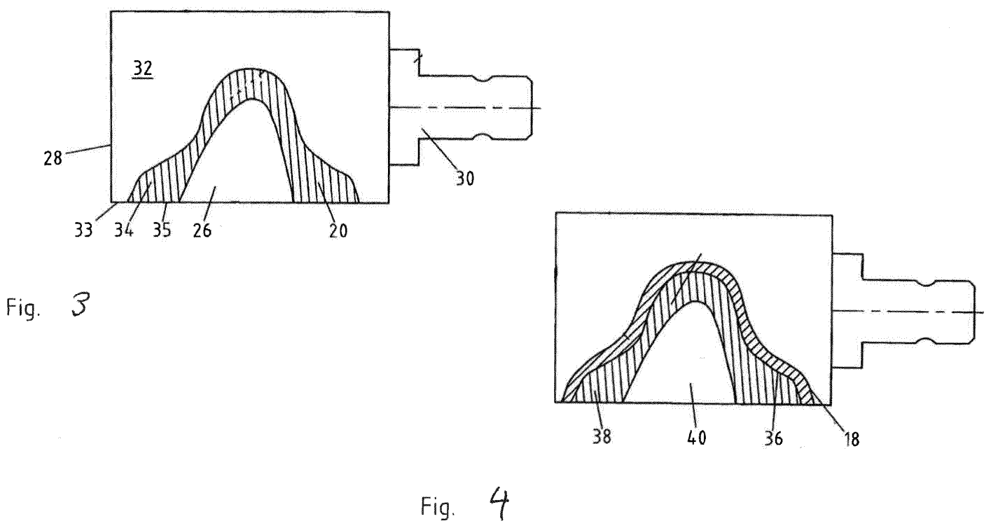

FIG. 4 shows that after filling the first cavity 18 in the first ceramic material 14 and filling the second ceramic material 20 into the cavity 18, a second cavity 36 is introduced, if necessary according to the method according to FIG. 2b, in order to then introduce into the cavity 36 thus formed a third ceramic material 38 that differs from the second ceramic material in its composition in such a way that, in particular, a higher strength can be achieved.

A cavity 40 can also be formed in the third ceramic material 38, as explained in connection with FIG. 3.

FIG. 5 illustrates how a dental restoration, a tooth 42 in the exemplary embodiment, is machined from the blank 28. For this purpose, after knowing the course of the first region 32 made of the first ceramic material 14 and the second region 34 made of the second ceramic material 20 in the blank 28, the tooth 42 to be produced is placed in the regions 32, 34 virtually in such a way that the cutting edge runs in the first region 32 and the dentine runs in the second region 34.

After the tooth 42 that has been virtually positioned in this way has been machined from the blank 28, a dental prosthesis is available that, in principle, can be used immediately and, in particular, does not require a veneer. A monolithic tooth is produced. In this case, the machining of the blank is facilitated by the fact that the second region 34 already has an open cavity 26, as explained in connection with FIG. 3 and can be seen in FIG. 5.

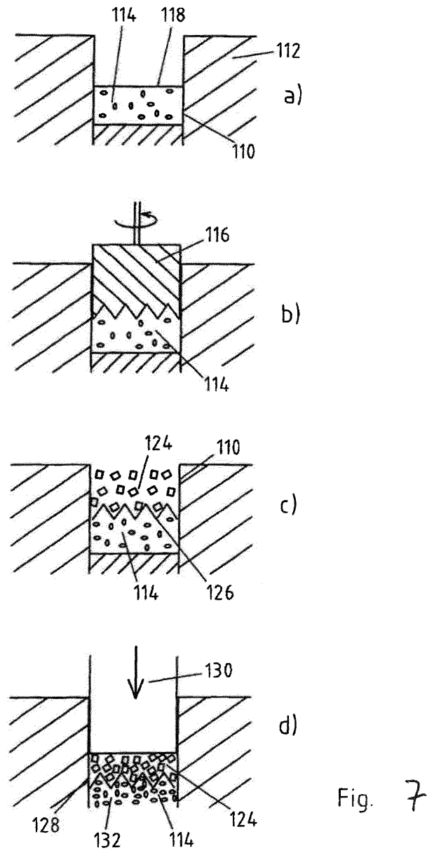

As can be seen in FIG. 6, there is also the possibility of forming a blank 28 with a large number of regions 52, 54, 56, which consist of the second and optionally a third ceramic material and can have different geometries in order to be able to produce corresponding teeth of different geometries. The so-called second regions 52, 54, 56 formed from the second ceramic material 20 are embedded in the first ceramic material, i.e. surrounded by it, as can be seen in the drawing.

If a dental product is preferably machined from the pre-sintered blank or optionally from the green body before the blank is fully sintered, then a departure from the invention of course does not result if significant machining takes place only after the blank has been completely sintered.

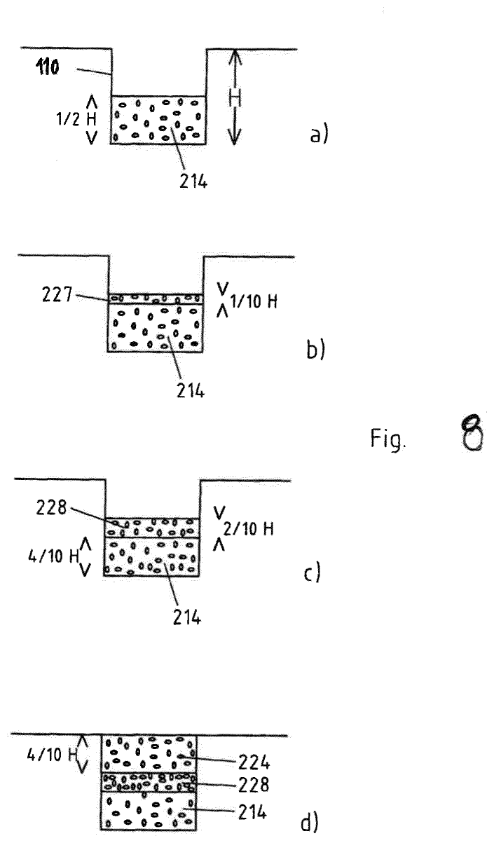

Further embodiments of the teaching according to the invention result from FIGS. 7 and 8. In order to produce a blank that consists of regions or layers of different compositions, wherein a continuous transition in terms of the desired properties are achieved between the layers. i.e. in the case of a tooth to be produced, for example, the incisal region is more translucent than the dentine region and the strength in the dentine region is greater than in the incisal region, a first material (layer 114) is filled into a die 110 of a press 112, which contains powdered starting materials, wherein zirconium dioxide is the base material, which is stabilised with at least gadolinium(III) oxide as the primary stabiliser and optionally with one or more secondary stabilisers. To the extent necessary, one or more colouring oxides and/or elements showing fluorescence properties are mixed. Binder can also be added.

After filling the first material into the die 110, the surface is smoothed and structured according to FIG. 7b. For this purpose, an element 116 in the form of a disc, plate or web is used, which in the exemplary embodiment has a serrated geometry on the layer side, so that a corresponding negative structure is formed in the surface 118 of the layer 114 by displacing material. This structure appears as concentric elevations and valleys surrounded by them. The structure should be designed in such a way that the volume of the elevations is equal or approximately equal to the volume of the depressions or valleys. According to FIG. 7c, a layer 124 consisting of a second material is then filled into the die 110, which differs from the first material with respect to the content of a stabiliser or the composition of the stabilisers or, in the case of a constant content of stabiliser(s), with respect to the other components.

The fact that the material of the second layer 124 penetrates to the bottom of the valleys 126 in the surface 118 of the layer results in a continuous transition between the properties of the layer 114 and the layer 124 after the layers 124, 114 have been pressed according to FIG. 7d. The transition layer is indicated with 128 in FIG. 7d.

As noted, the layer 124 is made of a different material than the layer 114. The deviation should deviate in particular in the colour additives and when one or more elements generating fluorescence are mixed in, in their proportions.

Regarding possible deviations in the stabilisers, it should be noted that the second layer 124 should be less in the proportion of the tetragonal crystal phase in the layer 124 than in the layer 114.

An alternative method for producing a so-called 2D blank, i.e. one in which no regions are formed that are contour-matched to the dental product to be produced, as explained with reference to FIGS. 2 to 6 and therefore the blanks can be referred to as 3D blanks, can be seen in FIG. 8.

A first ceramic material in powder form is thus introduced into a die 110, which can correspond to that of the layer 114 according to FIG. 7. The corresponding layer is indicated with 214 in FIG. 8a. The height of this layer 214 can be half the height of the total layers that are introduced into the die 110.

A layer 227 is then applied to the layer 214 with a thickness that is, for example, 1/10 of the total height of the layers. The material of the layer 227 can correspond to that of the second layer 124 according to FIG. 7. The layer 227 then is then mixed with a surface portion of the layer 214 over a depth that should correspond to the thickness of the layer 227. This forms an intermediate layer 228 that, as indicated above, has a thickness of 2/10 of the total height of the layers. A further layer 224 that corresponds to the second layer 124 according to FIG. 7 is then applied to the intermediate layer 228.

The height of the layer 224 in the exemplary embodiment is therefore 4/10 of the total height H. Subsequently, the layers 224, 228, 214 are pressed as a whole in order to then carry out the method steps of pre-sintering, machining and sintering (fully sintering), as has been explained. Machining after sintering can of course also take place.

1-15. (canceled)

16. A blank for use in producing a dental product, the blank comprising zirconium dioxide (ZrO₂) in a tetragonal phase, wherein the zirconium oxide is stabilised with gadolinium(III) oxide (Gd₂O₃) as a main stabiliser and with at least one oxide selected from the group comprising Y, Yb, Dy, Nd, Ca, Ce, Mg, Sm, Er, Tb, and La as a secondary stabiliser, wherein the secondary stabilizer is present in an amount of between 0.2 and 0.8 mol % relative to the content of ZrO₂, and the tetragonal phase of the zirconium oxide is present in an amount of between 40 and 80 vol %, and wherein the content of Gd₂O₃ is between 2.5 and 4 mol % relative to content of ZrO₂.

17. The blank according to claim 16, characterised in that the blank further comprises at least one colouring oxide from the group comprising Pr, Er, Fe, Co, Ni, Ti, V, Cr, Cu, Mn, and Tb.

18. The blank according to claim 16, characterised in that the tetragonal phase of the zirconium dioxide is present in an amount of between 45 and 75 vol %.

19. The blank according to claim 17, characterised in that the proportion of the at least one colouring oxide in the blank is at most 1.5 wt %.

20. The blank according to claim 16, characterised in that the blank has at least two regions with differing compositions, wherein either the total content of the stabilisers is the same in each region, or the total content of the stabilisers in the regions and/or the composition of the stabilisers is different.

21. The blank according to claim 16, characterised in that the blank or a region of the blank contains at least one element that produces a fluorescence effect, comprising at least one oxide from the group comprising Bi, Tb, Tm, and Pr, in a proportion of between 0.005 and 2.0 wt %.

22. The blank according to claim 16, characterised in that a first region has a cavity, within which a second region extends, which has a composition that differs from the first region, wherein the first region optionally has a plurality of cavities, optionally of different internal geometry, in which a plurality of second regions extend, and in that optionally the first region has a greater translucency than the second region, the strength of which is greater than that of the first region.

23. The blank according to claim 16, characterised in that the blank has at least three layers, wherein a middle layer is located between an upper and a lower layer, the middle layer comprising a material of the upper layer and the lower layer.

24. The blank according to claim 16, characterised in that the blank is multi-layered, comprising at least one bottom layer and one top layer of different compositions, wherein the layers contain at least one first colouring oxide, the proportion of which in the bottom layer having the first colouring oxide is lower than in the top layer having the first colouring oxide, wherein the first colouring oxide comprises at least one oxide from the group comprising Co, Mn, Ni, and Cr.

25. The blank according to claim 16, characterised in that the ratio of the main stabiliser Gd₂O₃ to the secondary stabiliser or the sum of the stabilising secondary stabilisers is 1:1 to 1:120.

26. A method for producing a blank intended for producing a dental product, which comprises the steps of a) pressing a powdered ceramic material containing zirconium dioxide stabilised with at least one stabilizer, and b) subjecting the pressed material to at least one heat treatment, wherein the zirconium dioxide contains a tetragonal phase and is stabilised with gadolinium(III) oxide (Gd₂O₃) as a main stabiliser and at least one oxide from the group comprising Y, Yb, Dy, Nd, Ca, Ce, Mg, Sm, Er, Tb, and La as a secondary stabilizer, and wherein the powdered ceramic material comprises between 2 and 5 mol % of gadolinium(III) oxide (Gd₂O₃) and between 0.2-0.8 mol % of the secondary stabiliser, relative to the amount of ZrO₂, and wherein the amount of the tetragonal phase of zirconium oxide is between 40 and 80 vol %.

27. The method according to claim 26, characterised in that at least one colouring oxide from the group comprising Pr, Er, Fe, Co, Ni, Ti, V, Cr, Cu, Mn, and Tb is added to the ceramic material.

28. The method according to claim 26, characterised in that a layer of a first ceramic material of a first composition is introduced into a die, in that at least a first open cavity is formed in the layer, into which a second ceramic material of a second composition is introduced, and in that the materials are pressed together and then heat-treated, wherein optionally after the second ceramic material has been introduced therein, a second open cavity is introduced into which a third ceramic material is introduced, the composition of which is different from that of the first and/or the second ceramic material, and in that optionally, a plurality of first open cavities, optionally of different geometries, are formed in the layer of the first ceramic material and the second ceramic material is introduced into these cavities.

29. The method according to claim 26, characterised in that, after a first layer of a first ceramic material has been introduced into a die the layer is structured on the surface in such a way that elevations and depressions bounded by these are formed, and a second ceramic material is subsequently introduced into the die, or in that, after the first layer has been introduced onto the latter, a further layer of a mixture that differs from the first layer is introduced into the die, in that the first layer is mixed in its surface region with the second layer to form an intermediate layer, and in that the second layer is then introduced into the die, wherein the structure is preferably produced by an element that moves, in particular rotates, relative to the first layer and that structures the surface region of the first layer with a wave-like, comb-like or sawtooth-like section, or in that preferably the structure is created with a pressure element acting in the direction of the surface of the first layer.

30. A dental product in the form of a dental restoration, or of an implant system comprising an implant and abutment, produced from the blank of claim 1, characterised in that in the case of a restoration, viewed in a direction of a tooth axis, it comprises at least a first layer running on a root side and a second layer running on an incisal side, wherein the strength of the first layer is greater than that of the second layer and the translucency of the second layer is greater than that of the first layer, and/or in that the layers contain a first colouring oxide from the group comprising Co, Mn, Ni, Cr, or mixtures thereof, wherein the proportion of the first colouring oxide in the first layer is lower than in the second layer.
